(19) United States
(12) Patent Application Publication
(10) Pub. No.: US 2002/0199220 A1
(43) Pub. Date: Dec. 26, 2002

(54) Methods to propagate plants via somatic embryogenesis and to transfer genes into ornamental statice and other members of the family plumbaginaceae

(76) Inventors: Bala Rathinasabapathi, Gainesville, FL (US); Mohammed A.M. Aly, Giza (EG)

Correspondence Address: LAW OFFICES OF BRIAN S STEINBERGER, 101 BREVARD AVENUE, COCOA, FL, 32922, US

(21) Appl. No.: 09/800,694

(22) Filed: Mar. 7, 2001

(51) Int. Cl. A01H 5/00

(52) U.S. Cl. 800/304

(57) ABSTRACT

Plumbaginaceae is a highly stress-tolerant angiosperm family consisting of approximately 15 genera of which the genus Limonium is the largest. Many species of Limonium (also called sea lavender and statice) are of commercial importance in ornamental horticulture, especially for cut flowers. It is taught herein that it is possible to generate Limonium plants via somatic embryogenesis and in turn provide a means for repetitive embryogenesis. This repetitive property allows perpetuation of the embryogenic state indefinitely and thereby produce large numbers of Limonium embryos for diverse goals such as mass propagation, production of valuable compounds and for the production of transgenic plants.

This invention relates to the propagation of plants and in particular to the propagation of ornamental statice via somatic embryogenesis and genetic transformation of ornamental statice.

### BACKGROUND AND PRIOR ART

Biotechnologies for the increased production of plants and for plant improvement are of intense interest because they provide a means of overcoming some of the traditional limitations of crop breeding and production. The two general approaches to plant propagation are organogenesis and somatic embryogenesis. The latter has been widely used because of its excellent potential for use in propagation and gene transfer systems (see W. A. Parrott et al, Ch.7 pp 158-200 of Murray D R (Ed) “Advanced methods in plant breeding and biotechnology”, CAB International, Wallingford, 1991). Somatic embryogenesis in its wide usage has been applied in the propagation of both moncot plants as bamboo and dicot plants such as sunflowers and soybeans.

Plumbaginaceae is a highly stress-tolerant angiosperm family consisting of approximately 15 genera of which the genus Limonium is the largest. Many species of Limonium (also called sea lavender and statice) are of commercial importance in ornamental horticulture, especially for cut flowers. This genus includes mainly rosulate plants with showy inflorescences. In the Iberian Peninsula, many of the Limonium species are endemic due to the stressful ecological conditions of their natural habitats (maritime cliffs and saline soils) and endangered because these habitats are under strong anthropogenic pressure.

The ability of Limonium species to adapt to abiotic stresses have provoked our research interest (see B. Rathinasabapathi et al, “Osmoprotectant β-alanine betaine synthesis in the Plumbaginaceae: S-adenosyl-L-methionine dependent N-methylation of β-alanine to its betaine is via N-methyl and N,N-dimethyl β-alanines.” Physiol Plant 109:225-231, 2000) since genes encoding this adaptation ability could potentially be used for improving crops for stress tolerance.

Plant regeneration via tissue culture, i.e, organogenesis and somatic embryogenesis, is an important tool in propagation, mutant selection and genetic transformation. A limited number of studies have focused on in vitro regeneration of plants in this Plumbaginaceae family (see Harazy et al, “In vitro propagation of statice (Limonium sinuatum) as an aid to breeding.” Hort Science 20:361-362, 1985) wherein L. sinuatum was propagated by culturing axillary buds. Similar meristem culture techniques were employed to propagate several species of Limonium [see: C. Martin et al, “Multiplication in vitro of Limonium estvei Fdez. Casa.” Annal Bot 70:165-167, 1992; C. Martin et al, “Micropropagation of five endemic species of Limonium from the Iberian Peninsula.” Jour Hort Sci 70:97-103, 1995; T. Matsumoto et al, “Induction of in vitro cultured masses of shoot primordia of hybrid statice and its cryopresevation by vitrification.” Hort Science 32:309-311, 1997; and, J. B. Amo-Marco et al, “Micropropagation of Limonium cavanillesti Erben, a threatened statice, from inflorescence stems.” Plant Growth Regulation 24:49-54, 1998].

Kunitake and Mii (see H. Kunitake et al, “Plant regeneration from cell culture-derived protoplasts of statice (Limonium perezii Hubbard).” Plant Sci 70:115-120, 1990 [hereinafter referred to as Kunitake and Mii]) regenerated plants from L. perezii suspension culture-derived protoptasts. This shoot regeneration via organogenesis was achieved according to this paper by culturing calli on media supplemented with a cytokinin.

Seelye et al. reported shoot regeneration from leaf discs of L. peregrinum, on media supplemented with either zeatin or thidiazuron (see J. Seeyle et al. “Shoot regeneration from leaf discs of Limonium perigrinum using thiadiazuron.” New Zealand Jour Crop and Hort Sci 22:23-29, 1944).

Many members of the Plumbaginaceae accumulate different nitrogenous osmoprotectants in response to stress (see Hanson et al, “Osmoprotective compounds in the Plumbaginacae; A natural experiment in metabolic engineering of stress tolerance.” Proc Natl Acad Sci USA 91: 361-362, 1994). One of the osmoprotectants, β-alanine betaine, is of pharmaceutical interest as a cholesterol-lowering compound (see K. Nisizawa, “Marine algae from a viewpoint of pharmaceutical studies.” Jap. J. Phycol 26:73-78, 1978). Plumbagin, a secondary product formed in many members of this family was reported to have anticancer properties (see U. Devi et al, “Plumbagin, a plant naphthoquionone with anti-tumor and radiomodifying properties.” Pharmaceutical Biology 37:231-236, 1999). After these pharmaceutical findings are medically confirmed, a method to produce large amounts of β-alanine betaine, plumbagin and other compounds will be needed.

However, none of these above referenced studies reported Limonium plant regeneration via somatic embryogenesis, which will provide a means for repetitive embryogenesis. This repetitive property would allow perpetuation of the embryogenic state indefinitely and thereby produce large numbers of uniform Limonium embryos for diverse goals such as mass propagation of rooted seedlings and the facile production of transgenic plants and eventual sources of pharmaceuticals.

### BRIEF SUMMARY OF THE INVENTION

The first objective of the present invention is to provide Plumbaginaceae plants via somatic embryogenesis.

The second object of this invention is to provide a method for somatic embryogenesis of Plumbaginaceae plants.

A third object of this invention is to provide a process suitable for the bioreactor production of Plumbaginaceae plants.

The fourth object of this invention is to provide a process to transfer genes into members of the Plumbaginaceae plants.

A preferred embodiment of the invention is Plumbaginaceae plants of genetic uniformity and a somatic embryogenetic method comprising the sequential steps of: first inducing Plumbaginaceae explants to initiate embryonic calli in from 7 to 10 days; then inducing maturation and germination of said calli within 14 to 30 days into somatic seedlings; and finally retrieving said somatic seedlings as rooted plants.

### BRIEF DESCRIPTION OF THE FIGURES

FIG. 1 is a series of 5 side views of the sequential process steps by which cultured vegetative tissue develops via the known organogenetic mode of regeneration first into shoots alone which shoots are then excised and rooted in a rooting medium.

FIG. 2 is a series of 4 side views of the sequential process steps by which cultured vegetative tissue develops via the somatic embryogenesis process wherein somatic embryos arise directly or indirectly from single cells and the embryos develop shoot and root axes simultaneously to provide uniform somatic seedlings.

### DESCRIPTION OF THE PREFERRED EMBODIMENT

Before explaining the disclosed embodiment of the present invention in detail it is to be understood that the invention is not limited in its application to the details of the particular arrangement shown since the invention is capable of other embodiments. Also, the terminology used herein is for the purpose of description and not of limitation.

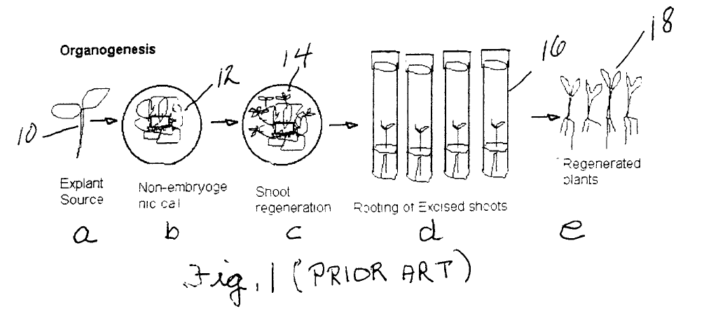

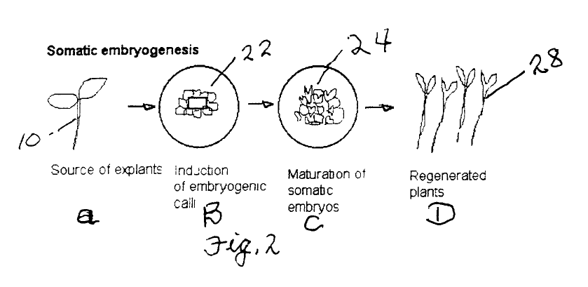

It would be useful to discuss the meanings of some words used herein and their applications before discussing FIGS. 1 and 2 including—

somatic embryogenesis Process by which embryos develop from cultured vegetative tissue. Somatic embryos arise directly or indirectly from single cells and the embryos develop shoot and root axes simultaneously.

Organogenesis Process by which cultured vegetative tissue develops either shoot or root. Organogenetic mode of regeneration usually involves several cells. In practice, shoots alone develop first and the shoots are excised and rooted in a rooting medium.

Explant is a portion of tissue used for tissue culture where the tissue can be seedling, plant, stem, and the like.

Plumbaginaceae Statice family

Limonium A genus within the Plumbaginaceae

Statice Older name for the genus Limonium

Sea lavender Common name for Limonium

Angiosperm Higher plants

Endosperm Part of a seed's storage tissue that results from the fusion of one of the male nucleus and two nuclei from the female

Meristem Undifferentiated tissue with highly dividing cells

Protoplast Cells without cell walls

Calli (singular Callus) Undifferentiated mass of proliferating cells

Cotyledon Embryonic leaf

Hypocotyl Stem portion of a seedling just below the embryonic leaves

Transgenic A plant expressing a foreign gene

Kinetin A cytokinin type plant growth regulator

Auxin A class of plant growth regulator. This is indole-3-acetic acid and its analogs.

Somatic seedling A young plant regenerated via somatic embryogenesis.

Referring now to FIG. 1, in the case of the known organogenetic mode of regeneration, the procedure is shown in a series of five steps (a-e). The explant 10 (oftentimes prepared from seeds) of step a is used for proliferation of a non-embryogenic callus mass 12 of step b by regeneration of shoots 14 in a medium suitable for the shoot regeneration of step c and then the shoots are excised and transferred to the root induction medium 16 of step d where they produce adventitious roots 18 as shown in step e.

On the other hand, in somatic embryogenesis, both the shoot and root meristems develop simultaneously from a somatic embryo (see FIG. 2) in four (not five) steps (a, B, C and D). The explant 10 (oftentimes prepared from seeds) of step a is used for induction of embryogenic calli 22 of step B followed by maturation of the somatic embryos 24 in a medium suitable for the maturation of step C wherein both the shoot and root meristems develop simultaneously into the regenerated markedly uniform plants 28 as shown in step D. Because of this simultaneous development of both the shoot and root meristems, and its single cell origin, propagation via somatic embryogenesis has the important advantages of uniform plant regeneration in a process involving fewer culture transfers resulting in economies of time, increased yields of somatic seedlings and regeneration in bio-reactors.

Example 1 discloses a method of producing seedlings (explants) to initiate embryogenic calli.

(1) Seed Sterilization and Germination to Prepare Explants

Seeds of perennial statice Limoniurn bellidifolium (Gouan) Durmort.(Statice caspia Willd.) and annual statice Linonium sinuatum (L.) Mill. Cv. Soiree, and other Limonium species were purchased from Park Seed Co. (Greenwood, S.C., U.S.A.). Seeds were surface-sterilized by rinsing in 70% (v/v) ethanol for 30 seconds and in 10% (v/v) commercial bleach for 10 minutes. Following this treatment, they were washed five times in sterile distilled water and germinated under light on half-strength Murashige and Skoog basal medium (MS, Murashige and Skoog, 1962) containing 1.5% (wt./v) sucrose and 0.8% (wt./v) agar (Agar-agar, Sigma, St. Louis, Mo., U.S.A.). The pH was adjusted to 5.8 prior to autoclaving for 5 days to provide the explants.

Example 2. Induction of embryogenic cultures.

Cotyledon and hypocotyl explants were dissected from 5-day old seedlings. The hypocotyls were cut 1 mm below the cotyledonary node and 1 mm above the hypocotyl-root junction to provide 2 mm-long explants. The cotyledons were separated from the cotyledon-petiole junction and halved into 2 to 3 mm long pieces. The explants were cultured, 10 per 100×15 mm Petri plate, on MS medium containing MS mineral salts, B5 vitamins (Gamborg et al. 1968), sucrose (2, 3 or 4% wt./v), 0.1 mg.l⁻¹ kinetin and 1, 2 or 5 mg.l⁻¹ 2,4-D, pH 5.8. The plates were sealed with parafilm (American National Can, WI) and incubated at 25° C. under 16/8 h (day/night) light regime (PPFD 46 μmol.quanta.m⁻².s⁻¹, cool white fluorescent lamps). The explants were subcultured every two weeks on the same media.

Cotyledon explants from L. bellidifolium and L. sinuatum initiated callus within 7-10 days in culture on media containing 1 mg.l⁻¹ 2,4-D and various levels of sucrose. Embryogenic callus was characterized by globular structures, smooth and shiny texture and creamy color. On media containing 1 mg.l⁻¹ 2,4-D and 0.1 mg.l⁻¹ kinetin, increasing sucrose concentrations increased the induction of well developed embryogenic calli, 4% sucrose being the optimum (see the following Table 1). Some of the cotyledon explants respond with direct somatic embryogenesis without a callus phase. The embryogenic calli produced repetitive globular embryos on the same induction media for both species. Medium containing 4% sucrose was also optimum to induce embryogenic calli from hypocotyl explants (79% response for 28 explants cultured for L. bellidifolium and 86% response for 21 explants cultured for L. sinuatum). On an average, 2-3 somatic embryos developed per explant weekly. At 2% sucrose, friable non-embryogenic callus grew along with less defined embryogenic calli on the same explant.

TABLE I
Response of L. bellidifolium and L. sinuatum cotyledon
explants to sucrose. Explants were scored for
the induction of embryogenic calli 30 days after culture.

| | | Number of | | | | |
|---|---|---|---|---|---|---|
| | | Explants | | | | |
| | Sucrose | responded/cultured | | | % | Callus |
| Species | (%)¹ | Exp.1 | Exp.2 | Exp.3 | Response | Description³ |
| L. bellidifolium | 2 | 15/24 | 23/28 | 53/67 | 75 | W,E,F |
| | 3 | 46/58 | 53/62 | 48/68 | 79 | W/G,E(g,t) |
| | 4 | 29/32 | 1/26 | 39/41 | 89 | W/G,E(g,h,t) |
| L. sinatum | 2 | 12/19 | 38/44 | 13/20 | 72 | W,E,F |
| | 3 | 50/61 | 49/74 | 61/69 | 79 | W/G,B(g,t) |
| | 4 | 42/48 | 32/39 | 37/40 | 88 | W/G,B(g,h,t) |

¹MS medium supplemented with 1 mg.1⁻¹ 2,4-D, 0.1 mg.1⁻¹ kinetin and sucrose as indicated.
²Mean + Standard error are for % response from two or three experiments (Exp.1 to 3).
³Callus description: W--white, G = green, E = embryogenic, F = friable, g = globular, h = heart and t = torpedo.

Example 3 demonstrates somatic embryo maturation in a medium containing sucrose and kinetin.

For maturation, somatic embryos at late-globular, heart and torpedo stages were subcultured on MS media containing 3% or 4% (wt./v) sucrose with or without 0.1 mg.l⁻¹ kinetin. Calli differentiating globular embryos were transferred to maturation medium, MS supplemented with 3 or 4% sucrose and 0 or 0.1 mg.l⁻¹ kinetin. Late globular, heart, torpedo and cotyledonary stages differentiated in these media and with faster and higher response on media with 4% sucrose compared to that on 3% sucrose (see Table 2). On media containing 3% sucrose with no growth regulators, somatic embryos developed abnormally long roots with poor shoot development. Increasing the sucrose concentration to 4% inhibited this abnormal rooting and supported maturation of somatic embryos into seedlings with normal morphology.

Supplementation of this medium with 0.1 mg/l kinetin induced embryo maturation and germination within 10 to 14 days of subculture compared to more than 21 days on media lacking kinetin.

Somatic embryos at the cotyledonary stage were transferred to MS basal medium lacking sucrose, the germination medium. L. bellidifolium somatic embryos germinated after 2-3 days on MS basal media lacking sucrose and growth regulators, while L. sinuatum somatic embryos did not develop beyond the cotyledonary stage. Some L. bellidifolium somatic embryos germinated on the induction media and formed leafy, cotyledon. The leaves, stems and roots of these plants produced numerous de novo green somatic embryos, i.e. repetitive somatic embryogenesis.

TABLE 2
following shows the effect of sucrose and kinetin on maturation and
germination of somatic embryos from cultured embryogenic calli derived
from L. bellidifolium and L. sinuatum cotyledon and hypocotyl explants.

| | Kinetin (mg.1) | | | |
|---|---|---|---|---|
| | 0.0 | | 0.1 | |
| Treatment | Maturation | Germination | Maturation | Germination |
| 3% sucrose | + | − | ++ | ++ |
| 4% sucrose | ++ | + | ++++ | ++++ |

− = No response, + slow (21-30 days); ++ medium (15-20 days); ++++- Fast (14 days) with 100% maturation and germination.

It would be useful to point out that the process of Examples 3 and 4 for enhanced propagation of remarkably uniform somatic seedlings would be best carried out in one or more bioreacters. The referenced somatic embryogenesis process of said Examples comprises the sequential steps of: (a) first inducing Plumbaginaceae explants to initiate embryonic calli; (b) then inducing maturation and germination of said somatic embryos into somatic seedlings; and (c) finally retrieving said somatic seedlings as rooted plants wherein a bioreactor is used for at least one of the steps (a) and/or (b) and under the physical and chemical conditions taught herein. It is preferred to use the bioreacter for the induction step (b).

Kunitake and Mii reported plant regeneration of Limonium perezii using Murashige and Skoog's medium. Their protocol also involved the use of 2,4-D but Kunitake and Mii did not report somatic embryogenesis. The reported process involved: multiplication of shoot apices in MS medium supplemented with 0.3 mg/l benzyladenine and 3% sucrose; followed by induction of calli on MS medium supplemented with 1 mg/l 2,4 D and 3% sucrose; and then initiation of suspension cell culture in MS medium supplemented with 1 mg/l 2,4-D and 3% sucrose liquid medium to provide protoplasts which were then isolated from the cell culture cells and calli were multiplied on MS medium containing 1 mg/l 2,4-D and 3% sucrose. These calli were regenerated in growth regulator-free MS medium or MS medium supplemented with zeatin. They observed separate adventitious roots and adventitious shoots in this regeneration medium. Following shoot proliferation, excised shoots were rooted in a separate rooting medium which conforms to step d of FIG. 1 which describes an organogenesis regeneration mode [regeneration proceeded with separate developments of shoots and roots].

Plant regeneration described in the present invention is the first to describe somatic embryogenesis of plants of the Plumbaganace family. The regeneration achieved in the present invention utilizes cotyledon and hypocotyl explants as opposed to protoplasts derived from tissue with a complex culture history as described by Kunitake and Mii whose culture duration from protoplasts to callus proliferation also takes 60 to 70 days, compared to a week in the method of the invention described herein.

Example 4 discusses the plant acclimatization of somatic seedlings to take them to the greenhouse.

Somatic seedlings developed via somatic embryogenesis were transferred to sterile potting medium in Magenta™ vessels (3″×3″×4″, Sigma Chemical-Company, USA), irrigated with Hoagland's nutrient solution, acclimatized by gradually lowering the humidity over 14 days and transferred to the greenhouse for propagation and if desired, analysis or genetic transformation.

Example 5 discusses cell suspension culture to ascertain if β-alanine betaine is produced in a cell culture.

Embryogenic L. latifolium liquid suspension culture was initiated from cotyledon and hypocotyl explants and cotyledon-derived calli induced on agar medium. MS medium (20 ml) supplemented with 1 mg.l⁻¹ 2,4-D and 2% (wt/v) sucrose was placed in 125 ml flasks. Cultures were incubated under diffuse light (PPFD 12 μmol.quanta.m⁻².s^{-1,} cool white flourescent lamps) in an orbital shaker at 125 rpm and subcultured every two weeks. To investigate whether the cell suspension cells are capable of synthesizing β-alanine betaine, radiolabeled formate (C¹⁴-formate of known specific activity) was supplied to 300 mg of fresh weight cells and incubated for 12 hr. The cells were extracted in a methanol:chloroform:water mixture and the aqueous fraction was further separated using ion exchange methods. The betaine fraction eluted from AG-50 ion-exchange column was analyzed using thin layer chromatorgraphy and autoradiography. The radioactive spot co-migrated with that of authentic β-alanine betaine.

Example 6 discloses the histology of our plant regeneration to show that it is by somatic embryogenesis

L. bellidifolium cotyledon explants cultured for two weeks, suspension cells cultured for eight weeks and individual somatic embryos were fixed in 4% (v/v) formaldehyde and 1% (v/v) gluteraldehyde in 100 mM sodium phosphate buffer, pH 7. The samples were post-fixed in 1% (v/v) osmium tetroxide and dehydrated in a series of ethanol solutions. Embedding was in Spurr's standard resin. Serial sections of 0.5 or 1 μm thickness were stained with Toluidine Blue and observed under a bright field microscope (Olyrnpus BH2-RFCA). Some of the Toluidine Blue stained sections were destained by soaking the sections in ethanol for 12 h, remounted and stained with Calcofluor-White ST (0.1% wt./v) and examined under fluorescence field (305 nm).

Histological observations of cultured L. bellidifolium cotyledon explants indicate that the proembryonal cell complexes arise from parenchyma cells immediately below the epidermal layer. Many more embryos developed on the side of the explant which was away from the medium than the side that was in contact with the medium. Globular embryos were rich in cytoplasm with small vacuoles while the non-embryogenic cells were highly vacuolated. Somatic embryos at various states of development were identified in suspension cultures. The histological observations confirm that plant regeneration observed is via somatic embryogenesis rather than organogenesis.

Example 7 discloses genetic transformation of Limonium species using the Agrobacterium-mediated gene transfer method.

The gene of interest (GOI) is cloned into a plant expression vector and the expression vector is transferred into the plant tissue using Agrobacterium tumefaciens. In this Example, a Ti-plasmid vector which contains right and left border sequences, NPTII with NOS promoter to confer kanamycin resistance to the transformed tissue and green fluorescent protein (GFP) which is a visual marker to identify transformed plants was employed. The GFP is under the control of CaMV 35S promoter.

Cotyledons and hypocotyl segments are dissected from young in vitro germinated seedlings. The explants are immersed in Agrobacterium-containing somatic embryogenesis induction medium (O.D 600=0.3-0.8) for 10 min. with shaking at 100 rpm, blotted on sterile filter paper and cultured on agar solidified embryogenesis induction medium for 3 days (Cocultivation). The explants are blotted on sterile filter papers, transferred to fresh medium containing 100 mg/l kanamycin sulfate, 250 mg/l carbenecillin and 500 mg/l Cefotaxime, continued culture under the same conditions mentioned above, and subcultured every 10 days. After 30 days on the antibiotic medium, where Agrobacterium growth had completely ceased on the explants and the medium, the explants are transferred to fresh medium containing 100 mg/l kanamycin to select the putative transformant embryogenic calli and/or somatic embryos.

The Agrobacterium does not exhibit growth on the antibiotic containing media within 5-7 days of culture. However, the explants are continued on this medium to ensure ridding the explants of any bacteria. After one week of culture on the antibiotics-containing medium, the explants exhibit spots (1 or more cells) with green fluorescence when exposed to UV light with 490 nm and 515 nm filter block while the uninfected control explants did not. While the control explants proved in every experiment that they do not tolerate kanamycin at 25, 50, 100 mg/l, a certain percentage of the infected explants form embryogenic calli on medium with 100 mg/l Kanamycin. Embryos at the early stages of development (e.g. globular and torpedo) are transferred to maturation medium (with no 2,4-D) containing kanamycin. Here seedlings develop further.

These putative transformants are analyzed for the presence and expression of the transferred genes by using the following techniques: (1) PCR amplification using genomic DNA as a template to identify the presence of a DNA sequence that is unique to the transferred DNA; (2) Southern blot hybridization to confirm the presence of the foreign gene; (3) RNA blot hybridization to identify the expression of mRNAs specific to the transferred gene and (4) Analyses to identify proteins (e.g. NPTII, GFP) encoded by the transferred genes. Efficiency of our transformation technique is scored by comparing the number of transformants obtained to the number of explants used in each experiment.

Although, marker genes are shown to be transferred in the example above, the technique is applicable to transfer any DNA sequence of interest. Depending upon the gene transferred, there is potential to obtain transgenic plants (1) altered in their flower colors; (2) with increased disease resistance (e.g. for Botrytis rot or viral diseases); (3) with long flower stems, (4) with attractive plant forms and (5) with resistance to a herbicide. Many of these trait modifications via gene transfer are known in prior art for other crops but have not been applied to improving Statice plants.

Specific Features of the Current Invention

Induction and maturation of somatic embryos in L. bellidifolium and L. sinuarum were influenced by 2,4-D and sucrose levels in the medium. Though MS medium supplemented with 2% sucrose resulted in embryogenic calli at relatively high frequencies (Table 1), it is not recommended because the callus mass was a mixture of friable and embryogenic cells.

Moreover, within the same culture intervals, the embryogenic calli reached only the globular stage while on induction media with 3 or 4% sucrose calli reached heart to cotyledonary stages (Table 1). Somatic embryogenesis in several other species are known to be influenced by sucrose and auxin levels. However, the protocol used here involves much lower concentration of 2,4-D than other studies and the induction is achieved rapidly. This is advantageous since high concentrations of 2,4-D and long culture periods could increase somaclonal variation.

Although plant regeneration was achieved in certain Limonium species, this is the first report of plant regeneration via somatic embryogenesis in any member of the Plumbaginaceae. Histological observations confirmed that the regeneration route is via somatic embryogenesis and the developmental sequence in L. bellidifolium is typical of somatic embryogenesis in other dicot angiosperms.

The results of our invention have important implications for propagation and improvement of ornamental statice. Since seeds of many Limonium species germinate poorly and have limited shelf life, tissue culture regeneration is an appealing alternative for propagating disease-free plants of high-quality statice. Somatic embryogenesis is preferred to organogenesis for micropropagation since it involves less culture intervals, less possibilities of somaclonal variation and is more amenable for automation and production of artificial seeds in bio-reactor vessels. Repetitive somatic embryogenesis is also ideal for genetic transformation since non-chimeric transformants could be regenerated and multiplied

The results of the several Examples show that the somatic embryogenesis of the Plumbaginaceae family of plants constitutes a means by which: 1) one can produce physical and genetically uniform plants; 2) much reduce the time for propagation of plants since the induction period is straight forward and of only about seven days with a total of 20 to 30 days to germinate somatic embryos as contrasted with when organogenesis is utilized; 3) one can achieve a repetitive somatic embryogenesis system which can produce plants at a frequency of about one million plants per explant per year thus providing for mass propagation of these species; 4) one can genetically transfer desired commercial and industrial traits to members of the Plumbaginaceae family such as variation in flower colors, resistance to disease such as viral diseases and Botrytis wilt and 5) one can achieve meaningful production level of osmoprotective and other useful compounds since the process is highly suitable for bioreactors.

The market is particularly in need of this technology to develop mass production of ornamental flowers, diversity in the varieties of cut flowers, an effective alternative to the use of chemical biocides by planting cultivars that are resistant to plant diseases and insect pests; and, to provide cell cultures of the Plumbaginaceae family for bioreactor production of useful compounds.

While the invention has been described, disclosed, illustrated and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

We claim:

1. A Plumbaginaceae family germinated seedling from a somatic embryo.

2. A seedling according to claim 1 wherein said embryo is provided solely by somatic embryogenesis and said family member is Limonium bellidifolium.

3. A seedling according to claim 1 wherein said embryo is provided solely by somatic embryogenesis and said family member is Limonium sinuatum.

4. A method of producing Plumbaginaceae plants by somatic embryogenesis comprising the sequential steps of:
(a) first inducing Plumbaginaceae explants to initiate embryonic calli in from 7 to 10 days;
(b) then inducing maturation and germination of said explants within 14 to 30 days into somatic seedlings; and,
(c) finally retrieving said somatic seedlings as rooted plants.

5. A method according to claim 4 in which maturation is in a medium containing approximately 4% by weight of sucrose and approximately 0.1 mg.l⁻¹ of kinetin.

6. A method according to claim 4 in which germination is in a medium containing approximately 4% by weight of sucrose and approximately 0.1 mg.l⁻¹ of kinetin.

7. A method according to claim 4 wherein a bioreactor is used for at least one of the steps (a) and (b)

8. A method according to claim 5 wherein at least one of said explants produces plants at a frequency of about one million plants per explant per year.

9. A method according to claim 7 wherein a bioreactor is used for step (b)

10. A method of introducing genetic transformation of a member of the Plumbaginaceae family comprising the steps of:
(a) preparing an Agrobacterium tumefaciens strain containing a vector
(b) infecting the tissue with said strain of (a); and,
(c) selecting transformed plants

11. A transgenic plant modified using the method described in claim 10.
